(19) United States
(12) Patent Application Publication
(10) Pub. No.: US 2012/0308525 A1
(43) Pub. Date: Dec. 6, 2012

(54) NUTRITIONAL COMPOSITIONS INCLUDING A HIGH PROTEIN COMPONENT AND EXOGENOUS NUCLEOTIDES

(75) Inventors: Norman Alan Greenberg, New Hope, MN (US); Kevin Burke Miller, Minneapolis, MN (US); Zamzam (Fariba) Kabiry Roughead, Plymouth, MN (US)

(73) Assignee: NESTEC S.A., Vevey (CH)

(21) Appl. No.: 13/510,517

(22) PCT Filed: Nov. 19, 2010
(86) PCT No.: PCT/US10/57310

§ 371 Date: Jul. 10, 2012

Related U.S. Application Data
(60) Provisional application No. 61/264,405, filed on Nov. 25, 2009.
(60) Provisional application No. 61/392,699, filed on Oct. 13, 2010.

(51) Int. Cl. A61K 38/02; A61P 3/02; A61P 31/00; A61P 7/02; A61K 33/00; A61K 35/74; A61K 36/815; A61K 36/886; A61K 35/12; A61P 17/02; A61P 7/00

(52) U.S. Cl. 424/93.4; 514/5.5; 424/725; 424/744; 424/537; 424/600

(57) ABSTRACT

Nutritional compositions and methods of making and using the nutritional compositions are provided. In a general embodiment, the present disclosure provides a nutritional composition including a high protein component and one or more exogenous nucleotides. The nutritional compositions can be specifically used to accelerate and improve wound healing in a mammal.

### BACKGROUND

The present disclosure generally relates to health and nutrition. More specifically, the present disclosure relates to nutritional compositions including protein and an exogenous nucleotide and methods of making and using the nutritional compositions.

There are many types of nutritional compositions currently on the market. Nutritional compositions can be targeted toward certain consumer types, for example, young, elderly, athletic, etc., based on the specific ingredients of the nutritional composition. Nutritional compositions can also be formulated based on the certain physiological conditions that the nutritional compositions are intended to treat or improve.

Patients are more susceptible to wound during care in the health system when they are malnourished as compared to free living individuals. These patients typically do not consume adequate amounts of protein. Despite the consumption of protein containing products, the prevention of wounds, such as pressure ulcers, is often not successful. Nutritional products can be used as part of a therapy to help heal and close the wounds. However the use of these nutritional products does not ensure a successful rapid healing of the various types of wounds. Furthermore, medical conditions such as diabetes will also interfere with wound healing.

It is estimated that 8-12% of all hospital patients will acquire a pressure ulcer in the first two weeks after admission. Even more long term care residents acquire pressure ulcers. Pressure ulcers are divided into four categories with stage I being the mildest and stage IV the most severe. The average time to heal these is 4 and 22 weeks for stage I and IV, respectively. This indicates that the current nutritional options for patients with pressure ulcers are not adequate to support rapid healing.

### SUMMARY

Nutritional compositions having a high protein component and an exogenous nucleotide and methods of making and using the nutritional compositions are provided. In a general embodiment, the present disclosure provides a nutritional composition comprising a high protein component and one or more exogenous nucleotides.

In an embodiment, the high protein component includes one or more proteins, peptides, and amino acids, and precursors and metabolites of proteins, peptides, and amino acids in an amount that provides at least about 18% of the total calories of the nutritional composition.

In an embodiment, the nutritional composition has a total volume of 300 mL, or less. The high protein component in this nutritional composition includes one or more proteins, peptides, and amino acids, and precursors and metabolites of proteins, peptides, and amino acids in an amount greater than about 14 grams.

In an embodiment, the high protein component includes at least about 5 grams of protein, in least about 3 grams of as bitter tasting amino acid, and at least about 7 grams of a neutral tasting acid. The bitter tasting amino acid can be arginine, phenylalanine, tyrosine, leucine, isoleucine, valine, methionine, histidine or a combination thereof. The neutral tasting acid can be glutamine, glycine alanine, threonine, praline, serine or a combination thereof.

In an embodiment, the exogenous nucleotide is in an amount of about 1 gram/1000 calories of the nutritional composition. The exogenous nucleotide can be in a monomeric form such as 5′ Adenosine Monophosphate, 5′-Guanosine Monophosphate, 5′-Cytosine Monophosphate, 5′-Uracil Monophosphate, 5′-Inosine Monophosphate, 5′-Thymine Monophosphate or a combination thereof. The exogenous nucleotide can be intact ribonucleic add or other compounds containing nucleotides.

In an embodiment, the nutritional composition further comprises one or more ingredients such as non-replicating bacteria, fatty acids, triglycerides, lactowolfberry, antioxidants, vitamins, minerals, polyphenolics flavonoids, EGCg, pycnogenol, α- and β-glucans, alpha-hydroxyisocaproate, aloe, honey, amino acids, branched chain amino acids or a combination thereof.

In an embodiment, the nutritional composition is in an administrable form such as pharmaceutical formulations, nutritional formulations, dietary supplements, functional foods and beverage products.

In another embodiment, the present disclosure provides a method of making a nutritional composition. The method comprises adding a high protein component and an exogenous nucleotide to a nutritional composition.

In an alternative embodiment, the present disclosure provides a method of improving wound healing in a mammal. The method comprises administering to a mammal having a wound a nutritional composition including an effective amount of a high protein component and one or more exogenous nucleotides. The nutrition composition can be administered to provide the exogenous nucleotide in an amount ranging from about 0.2 g/day to about 4 grams/day. The wound can be from pressure ulcers, surgical incisions, cuts, scrapes or a combination thereof. The mammal can also have diabetes.

An advantage of the present disclosure is to provide an improved nutritional composition having a high protein component and exogenous nucleotides.

Another advantage of the present disclosure is to provide a method of making an improved nutritional composition.

Yet another advantage of the present disclosure is to provide a nutritional composition that accelerates or improves wound healing in a mammal.

Additional features and advantages are described herein, and will be apparent from the following Detailed Description.

### DETAILED DESCRIPTION

The present disclosure relates to nutritional compositions including a high protein component and exogenous nucleotides and methods of making and using the nutritional corn positions. Embodiments of the nutritional compositions of the present disclosure can provide improved healing of wounds. The wounds may be of any type including pressure ulcers, accident injury, surgical incisions, etc. The need to improve wound healing is evident from the high cost of wound care, longer hospital stays, and morbidity experienced by patients.

As used herein, the term “nutritional composition” includes, but is not limited to, complete nutritional compositions, partial or incomplete nutritional compositions, and disease or condition specific nutritional compositions.

As used herein, “about,” is preferably understood to refer to numbers in a range of numerals. Moreover, all numerical ranges herein should be understood to include all integer, whole or fractions, within the range.

As used herein, “complete nutrition” are preferably nutritional products that contain sufficient types and levels of macronutrients (protein, fats and carbohydrates) and micronutrients to be sufficient to be a sole source of nutrition for the animal to which it is being administered to. Patients can receive 100% of their nutritional requirements from such complete nutritional compositions.

As used herein, “effective amount” is preferably an amount that prevents a deficiency, treats a disease or medical condition in an individual or, more generally, reduces symptoms, manages progression of the diseases or provides a nutritional, physiological, or medical benefit to the individual. A treatment can be patient- or doctor-related. In addition, while the terms “individual” and “patient” are often used herein to refer to a human, the invention is not so limited. Accordingly, the terms “individual” and “patient” refer to any animal, mammal or human having or at risk for a medical condition that can benefit from the treatment.

As used herein, “elderly” is preferably a human that is sixty-five years of age or older, more preferably seventy-five years or age or older.

As used herein, “incomplete nutrition” are preferably nutritional products that do not contain sufficient levels of macronutrients (protein, fats and carbohydrates) or micronutrients to be sufficient to be a sole source of nutrition for the animal to which it is being administered to. Partial or incomplete nutritional compositions can be used as a nutritional supplement.

As used herein, “Long term administrations” are preferably continuous administrations for more than 6 weeks.

As used herein, mammal, includes but is not limited to rodents, aquatic mammals, domestic animals such as dogs and cats, farm animals such as sheep, pigs, cows and horses, and humans. Wherein the term mammal is used, it is contemplated that it also applies to other animals that are capable of the effect exhibited or intended to be exhibited by the mammal.

As used herein the term “patient” is preferably understood to include an animal, especially a mammal, and more especially a human that is receiving or intended to receive treatment, as it is herein defined.

AS used herein, “phytochemicals” or “phytonutrients” are non-nutritive compounds that are found in many foods. Phytochemicals are functional foods that have health benefits beyond basic nutrition, and are health promoting compounds that come from plant sources. As used herein, “Phytochemicals” and “Phytonutrients” refers to any chemical produced by a plant that imparts one or more health benefit on the user. Phytochemicals can be administered by any means, including topically, enterally, and/or parenterally. As used herein, non-limiting examples of phytochemicals and phytonutrients include those that are:
1. Phenolic compounds which include Monophenols (such as Apiole, Carnosol, Carvacrol, Dillapiole, Rosemarinol); Flavonoids (polyphenols) including Flavonols (such as: Quercetin, Gingerol, Kaempferol, Myricetin, Rutin, Isorhamnetin), Flavanones (such as: Hesperidin, Naringenin, Silybin, Eriodictyol), Flavones (such as: Apigenin, Tangeritin, Luteolin), Flavan-3-ols (such as: Catechins, (+)-Catechin, (+)-Gallocatechin, (−)-Epicatechin, (−)-Epigallocatechin, (−)-Epigallocatechin gallate (EGCG), (−)-Epicatechin 3-gallate, Theaflavin Theaflavin-3-gallate, Theaflavin-3′-gallate, Theaflavin-3,3′-digallate Thearubigins), Anthocyanins (flavonals) and Anthocyanidins (such as: Pelargonidin, Peonidin, Cyanidin, Delphinidin, Malvidin, Petunidin), Isoflavones (phytoestrogens) (such as Daidzein (formononetin), Genistein (biochanin A), Glycitein), Dihydroflavonols, Chalcones, Coumestans (phytoestrogens), and Coumestrol, Phenolic acids (such as: Ellagic acid, Gallic acid, Tannic acid, Vanillin, Curcumin); Hydroxycinnamic acids (such as: Caffeic acid, Chlorogenic acid, Cinnamic acid, Ferulic acid, Coumarin); Lignans (phytoestrogens), Silymarin, Secoisolariciresinol, Pinoresinol and lariciresinol); Tyrosol esters (such as Tyrosol, Hydroxytyrosol Oleocanthal, Oleuropein); Stilbenoids (such as Resveratrol, Pterostilbene, Piceatannol) and Punicalagins;
2. Terpenes (isoprenoids) which include Carotenoids (tetraterpenoids) including Carotenes (such as: α-Carotene, β-Carotene, γ-Carotene, δ-Carotene, Lycopene, Neurosporene, Phytofluene, Phytoene), and Xanthophylls (such as: Canthaxanthin, Cryptoxanthin, Zeaxanthin, Astaxanthin, Lutein, Rubixanthin); Monoterpenes (such as Limonene, Perillyl alcohol), Saponins; Lipids including Phytosterols (such as: Campesterol, beta Sitosterol, gamma sitosterol, Stigmasterol), Tocopherols (vitamin E), and omega-3, 6, and 9 fatty acids (such as gamma-linolenic acid); Triterpenoid (such as Oleanolic acid, Ursolic acid, Betulinic acid, Moronic acid);
3. Betalins which include Betacyanins (such as betanin, isobetanin, probetanin, neobetanin); and Betaxanthins (non glycosidic versions) (such as: Indicaxanthin, and Vulgaxanthin);
4. Organosulfides which include Dithiolthiones (isothiocyanates) (such as: Sulphoraphane); and Thiosulphonates (allium compounds) (such as Allyl methyl trisulfide, and Diallyl sulfide), Indoles, glucosinolates which include Indole-3-carbinol; sulforaphane, 3,3′-Diindolylmethane; Sinigrin; Allicin; Alliin; Allyl isothiocyanate; Piperine; Syn-propanethial-S-oxide;
5. Protein inhibitors which include protease inhibitors;
6. Other organic acids which include Oxalic acid, Phytic acid (inositol hexaphosphate); Tartaric acid; and Anacardic acid; and
7. combinations thereof.

As used in this specification and the appended claims, the singular fonts “a”, “an” and “the” include plural referents unless the context clearly dictates otherwise. Thus, for example, reference to “a polypeptide” includes a mixture of two or more polypeptides, and the like.

As used herein, a “Prebiotic” is preferably a food substances that selectively promote the growth of beneficial bacteria or inhibit the growth of pathogenic bacteria in the intestines. They are not inactivated in the stomach and/or upper intestine or absorbed in the GI tract of the person ingesting them, but they are fermented by the gastrointestinal microflora and for by probiotics. Prebiotics are for example defined by Glenn R. Gibson and Marcel B. Roberfroid, Dietary Modulation of the Human Colonic Microbiota: Introducing the Concept of Prebiotics, J. Nutr. 1995 125: 1401-1412. Examples of prebiotics include acacia gum, alpha glucan, arabinogalactans, beta glucan, dextrans, fructooligosaccharides, galactooligosaccharides, galactomannans, gentiooligosaccharides, glucooligosaccharides, guar gum, inulin, isomaltooligosaccharides, lactosucrose, lactulose, leVan, maltodextrins, partially hydrolyzed guar gum, pecticoligosaccharides, retrograded starch, soyoligosaccharides, sugar alcohols, xylooligosaccharides, or a combination thereof.

As used herein, Probiotics micro-organisms (hereinafter “probiotics”) are preferably microorganisms (alive, including semi-viable or weakened, and/or non-replicating), metabolites, microbial cell preparations or components of microbial cell that could confer health benefits on the host when administered in adequate amounts, more specifically, that beneficially affect a host by improving its intestinal microbial balance, leading to effects on the health or well-being of the host. (Salminen S, Ouwehand A. Benno Y. et al “Probiotics: how should they be defined” Trends Food Sci, Technol. 1999:10 107-10). In general, it is believed that these micro-organisms inhibit or influence the growth and/or metabolism of pathogenic bacteria in the intestinal tract. The probiotics may also activate the immune function of the host. For this reason, there have been many different approaches to include probiotics into food products. Examples of probiotics include Aerococcus, Aspergillus, Bacteroides, Bifidobacterium, Candida, Clostridium Debaromyces, Enterococcus, Fusobacterium, Lactobacillus, Lactococcus, Leuconostoc, Melissococcus, Micrococcus, Mucor, Oenococcus, Pediococeus, Penicillium, Peptostrepococcus, Pichia, Propionibacterium, Pseudocatenulatum, Rhizopus, Saccharomyces, Staphylococcus, Streptococcus, Torulopsis, Weissella, or as combination thereof.

In a general embodiment, the present disclosure provides a nutritional composition comprising a high protein component and one or more exogenous nucleotides. The combination of a high protein content and one or more exogenous nucleotides can provide a synergistic benefit for accelerating or improving wound fielding in a mammal. As used herein, the term “mammal” refers to a human (e.g., child, adult) or an animal.

Nucleotides are low molecular weight biological molecules key to biochemical processes. Sources include de novo synthesis, recovery via salvage mechanisms, and dietary intakes. While endogenous production serves as the main nucleotide source, there is evidence to suggest that exogenous sources are important in rapidly proliferating cells in the immune and gastrointestinal systems, where they may become conditionally essential. Exogenous nucleotides may support optimal growth and function of metabolically active cells in times of cellular insult and their supplementation may improve clinical outcomes in the critically ill and immune suppressed patient.

The exogenous nucleotides can be in the form of monomers and polymers as part of the nutritional compositions. A nucleotide is a subunit of deoxyribonucleic acid (“DNA”) or ribonucleic acid (“RNA”). It is a organic compound made up of a nitrogenous base, a phosphate molecule, and a sugar molecule (deoxyribose in DNA and ribose in RNA). Individual nucleotide monomers (single units) are linked together to form polymers, or long chains. The exogenous nucleotides in embodiments of the present disclosure are specifically provided by dietary supplementation.

The exogenous nucleotides can be in a monomeric form such as, for example 5′ Adenosine Monophosphate (“5′-AMP”), 5′-Guanosine Monophosphate (“5″-GMP”), 5″-Cytosine Monophosphate (“5′-CMP”), 5′-Uracil Monophosphate (“5′-UMP”), 5′-Inosine Monophosphate (“5′-IMP”), 5′-Thymine Monophosphate (“5′-TMP”) or a combination thereof. The exogenous nucleotides can also be in a polymeric form such as for example, an intact RNA. There can be multiple sources of the polymeric form such as for example, yeast RNA. In an embodiment, the exogenous nucleotide is in an amount of about 1 gram/1000 calories of the nutritional composition.

In an embodiment, the high protein component includes one or more proteins, peptides, and amino acids, and precursors and metabolites of proteins, peptides, and amino acids in an amount that provides at least about 18% of the total calories of the nutritional composition.

in an embodiment, the nutritional composition has a total volume of 300 mL or less. The high protein component in this nutritional composition includes at least one of proteins, peptides, and amino acids, and precursors and metabolites of proteins, peptides, and amino acids in an amount greater than about 14 grams.

In an embodiment, the high protein component includes at least about 5 grains of protein, at least about 3 grams of one or more bitter tasting amino acids, and at least about 7 grams of one or more neutral tasting acids. The bitter tasting amino acid can be arginine, phenylalanine, tyrosine, leucine, isoleucine, valine, methionine, histidine or a combination thereof. The neutral tasting acid can be glutamine, glycine, alanine, threonine, praline, serine or a combination thereof.

The term “protein”, “peptide”, “oligopeptides” or “polypeptide” as used herein is preferably understood to refer to any composition that includes, a single amino acids (monomers), two or more amino acids joined together by a peptide bond (dipeptide, tripeptide, or polypeptide), collagen, precursor, homolog, analog, mimetic, salt, prodrug, metabolite, or fragment thereof or combination. For the sake of clarity, the use of any of the above terms is interchangeable unless otherwise specified. It will be appreciated that polypeptides (or peptides or proteins or oligopeptides) often contain amino acids other than the 20 amino acids commonly referred to as the 20 naturally occurring, amino acids, and that many amino acids, including the terminal amino acids, may be modified in a given polypeptide, either by natural processes such as glycosylation and other post-translational modifications, or by chemical modification techniques which are well known in the art. Among the known modifications which may be present in polypeptides of the present invention include, but are not limited to acetylation, acylation, ADP-ribosylation, amidation, covalent attachment of a flavanoid or a heme moiety, covalent attachment of a polynucleotide or polynucleotide derivative, covalent attachment of a lipid or lipid derivative, covalent attachment of phosphatidylinositol, cross-linking, cyclization, disulfide bond formation, demethylation, formation of covalent cross-links, formation Of cystine, formation of pyroglutamate, formylation, gamma-carboxylation, glycation, glycosylation, glycosylphosphatidyl inositol (GPI) membrane anchor formation, hydroxylation, iodination, methylation, myristoylation, oxidation, proteolytic processing, phosphorylation, prenylation, racemization, selenoylation, sulfation, transfer-RNA mediated addition of amino acids to polypeptides such as arginylation, and ubiquitination. The term “protein” also includes “artificial proteins” which refers to linear or non-linear polypeptides, consisting of alternating repeats of a peptide Non-limiting examples of proteins include dairy based proteins and plant based proteins. Dairy based proteins include casein, caseinates (e.g., all forms including sodium, calcium, potassium caseinates), whey (e.g., all forms including concentrate, isolate, demineralized), milk protein concentrate, and milk protein isolate. Plant based proteins include soy protein all forms including concentrate and isolate), pea protein (e.g., all forms including concentrate and isolate), canola protein (e.g., all forms including concentrate and isolate), other plant proteins that commercially are wheat and fractionated wheat proteins, corn and it fractions including zein, rice, oat, potato, peanut, and any proteins derived from beans, lentils, and pulses.

Additional examples of proteins can come from animal sources (e.g., meats, meat powders, organ meats, and extracts of the meats), marine sources (e.g., fish meal, fish muscle, and the same for all forms of marine animals), and microbial sources (e.g., single cell proteins and variations).

All of the proteins listed above can be used as intact proteins or as hydrolysates. The hydrolysates can have a low to high degree of hydrolysis. The peptides that result from the hydrolysis can include large or small peptides or a range of peptide sizes.

Free amino acids can be used in place of or in addition to proteins to provide the necessary amino acids. Dipeptides are also commercially available and can provide the necessary amino acids.

Non-limiting examples of peptides can range in size categories including small (e.g., di and tripeptides that require no additional breakdown for absorption in the intestine), medium (e.g., oligopeptides that are 4-10 amino acids long) large (e.g., >10 amino acids but smaller than small proteins—a molecular weight range of 1,000-5,000), and very large (e.g., molecular weights greater than 5,000 but not intake protein).

Additional ingredients may be used with the exogenous nucleotides and the high protein component to improve wound healing. In an embodiment, the nutritional composition further comprises one or more ingredients such as non-replicating bacteria, fatty acids, triglycerides, lactowolfberry, antioxidants, vitamins, minerals, polyphenolics, flavonoids, EGCg, pycnogenol, α- and β-glucans, alpha-hydroxyisocaproate, aloe, honey, amino acids, branched chain amino acids or a combination thereof.

Specific ingredients and their functions are described in more detail as follows:

Nonreplicating bacteria (e.g., inactivated bacteria)—support of immune function.

Arginine—precursor of proline via ornithine, precursor of nitric oxide for vasodilation, and immune function via T-cell stimulation.

Glutamine—precursor of arginine via citrulline; lymphokine activated killer (“LAK”) cell are dependent on glutamine for activity.

Fatty acids and triglycerides—modulation of inflammation and component of cell membranes and skin.

Lactowolfberry—Nestlé bioavailable form of goji berry for immune responsiveness.

Copper—essential for macrophage function and proliferation of immune cells in response to challenge.

Zinc—required for optimal innate immune response, essential co-factor for protein synthesis, cell division, and immune function.

Vitamin C—depleted by stress, needed for collagen synthesis, stimulation of interferon, maintenance of redox integrity of the cell and intracellular matrix components.

Vitamin A—important for regulation of antibody response as well as differentiation of B-cells to IgG-expressing cells also needed for skin maintenance and collagen cross linking.

β-carotene—precursor of Vitamin A, benefits same as for vitamin A plus antioxidant activity.

Vitamin E—lipid soluble antioxidant stabilizes cell membranes and lipids to prevent oxidative damage to immune cells.

Selenium—component of selenoproteins, augments cellular immune response through production of interferon, earlier peak of T cell proliferation, and increased T helper cells.

Polyphenolics—inhibit inflammatory cytokine, interleukin-1 and decrease production of prostaglandin E2.

Flavonoids—stimulate or inhibit protein phosphorylation and thereby regulate immune cell function.

EGCg—catechin with protective effects on dendritic cells that survey wound site, reduces the production of inflammatory mediators IL-6 and COX-2.

Pycnogenol—improves both T-cell and B-cell function and stimulates IL-2 and Natural Killer T-cell activity.

α- and/or β-glucans—immune cell stimulators of both innate and acquired immune systems via binding macrophages directly.

AHCC—(methylated α-glucan)—specifically modified alpha-glucan.

Aloe—suppression of inflammatory cytokines TNF-alpha and IL-1 beta.

Honey—osmotic and pH effects to prevent infection and immune stimulation.

Branched chain amino adds—readily oxidized and therefore supplemented for the synthesis or proteins by immune cells, such as protein antibodies (“Ab”) by B-cells.

Leucine—Most effective branched chain amino acid for the stimulation and maintenance of protein for immune cell proliferation kind muscle anabolism.

α-HICA—(α-Hydroxyisocitproic acid) metabolite of leucine that can improve anabolism.

In an embodiment, the nutritional composition further includes one or more amino acids. Non-limiting examples of amino acids include Alanine, Arginine, Asparagine, Aspartate, Citrulline, Cysteine, Glutamate, Glutamine, Glycine, Histidine, Hydroxyproline, Hydroxyserine, Hydroxytyrosine, Hydroxylysine, Isoleucine, Leucine, Lysine, Methionine, Phenylalanine, Proline, Serine, Taurine, Threonine, Tryptophan, Tyrosine, and Valine and combination thereof.

As used herein the term “antioxidant” is preferably understood to include any one or more of various substances (as beta-carotene (a vitamin A precursor), vitamin C, vitamin E, and selenium) that inhibit oxidation or reactions promoted by Reactive Oxygen Species (ROS) and other radical and non-radical species. Additionally, antioxidants are molecules capable of slowing or preventing the oxidation of other molecules. As used herein, non-limiting examples of antioxidants include carotenoids, coenzyme Q10 (“CoQ10”), flavonoids, glutathione Goji (Wolfberry), hesperidine, Lactowolfberry, lignan, lutein, lycopene, poly phenols, selenium, vitamin A, vitamin B1, vitamin B6, vitamin B12, vitamin C, vitamin D, vitamin E, and combinations thereof.

As used herein the term “vitamin” is preferably understood to include any of various fit-soluble or water-soluble organic substances (non-limiting examples include vitamin A, Vitamin B1 (thiamine), Vitamin B2 (riboflavin), Vitamin B3 (niacin or niacinamide), Vitamin B5 (pantothenic acid), Vitamin B6 (pyridoxine, pyridoxal, or pyridoxamine, or pyridoxine hydrochloride), Vitamin B7 (biotin), Vitamin B9 (folic add), and Vitamin B12 (various cobalamins; commonly cyanocobalamin in vitamin supplements) vitamin C, vitamin D, vitamin E, vitamin K (including Vitamin K1 and Vitamin K2), essential in minute amounts for normal growth and activity of the body and obtained naturally from plant and animal foods or synthetically made, pro-vitamins, derivatives, analogs. As used herein the term “minerals” is preferably understood to include boron, calcium, chromium, copper, iodine, iron, magnesium, manganese, molybdenum, nickel, phosphorus, potassium, selenium, silicon, tin, vanadium, and zinc.

As used herein, the terms “treatment”, “treat” and “to alleviate” is preferably to both prophylactic or preventive treatment (that prevent and/or slow the development of a targeted pathologic condition or disorder) and curative, therapeutic or disease-modifying treatment, including therapeutic measures that cure, slow down, lessen symptoms of, and/or halt progression of a diagnosed pathologic condition or disorder; and treatment of patients at risk of contracting a disease or suspected to have contracted a disease, as well as patients who are ill or have been diagnosed as suffering from a disease or medical condition. The term does not necessarily imply that a subject is treated until total recovery. The terms “treatment” and “treat” also refer to the maintenance and/or promotion of health in an individual not suffering front a disease but who may be susceptible to the development of an unhealthy condition, such as nitrogen imbalance or muscle loss. The terms “treatment”, “treat” and “to alleviate” are also intended to include the potentiation or otherwise enhancement of one or more primary prophylactic or therapeutic measure. The terms “treatment” “treat” and “to alleviate” are further intended to include the dietary management of a disease or condition or the dietary management for prophylaxis or prevention a disease or condition

As used herein a “tube feed” is preferably a complete or incomplete nutritional products that are administered to an animal's gastrointestinal system, other than through oral administration, including but not hunted to a nasogastric tube, orogastric tube, gastric tube, jejunostomy tube (J-tube), percutaneous endoscopic gastrostomy (PEG), port, such as a chest wall port that provides access to the stomach, jejunum and other suitable access ports.

In another embodiment, the present disclosure provides a method of making a nutritional composition. The method comprises adding a high protein component and one or more exogenous nucleotides to a nutritional composition in an effective amount, for example, to accelerate or improve wound healing in a mammal. The nutritional composition can be in an administerable form such as pharmaceutical formulations, nutritional formulations, dietary supplements, functional foods, beverage products or a combination thereof.

In an alternative embodiment, the present disclosure provides a method of improving wound healing in a mammal. The method comprises administering to a mammal having a wound a nutritional composition including an effective amount of a high protein component and an exogenous nucleotide. The nutrition composition can be administered to provide the exogenous nucleotide in an amount ranging from about 0.2 g/day to about 4 grams/day. The wound can be from pressure ulcers, surgical incisions, cuts, scrapes or a combination thereof. The mammal can also have diabetes.

In an alternative embodiment, the present disclosure provides at method of improving pressure ulcers in a mammal using the same steps previously described.

Other optional ingredients can be added to make the nutritional composition sufficiently palatable. The optional ingredients can be added in any suitable amount.

### EXAMPLES

By way of example and not limitation, the following examples are illustrative of various embodiments of the present disclosure.

### Example 1

TABLE 1
Complete feeding product appropriate for dietary management of
pressure ulcers

| Ingredient | Amount | | Function |
|---|---|---|---|
| Caseinate | 75 | g (20% of energy) | High quality protein |
| Canola oil | 50 | g (30% of energy) | Fatty acid source and |
| | | | energy |
| Maltodextrin | 188 | g (50% of energy) | Carbohydrate and energy |
| | | | source |
| Yeast extract | 2.5 | g | RNA/nucleotide source |
| Vitamin premix | 2.5 | g | |
| Mineral premix | 1.0 | g | |
| Emulsifier | 1.5 | g | |
| Water | 1290 | g | |
| Optional ingredients | | | As listed elsewhere |

### Example 2

TABLE 2
Formulation for dietary management of pressure ulcers, one day of feeding

| Ingredient | Amount | | Function |
|---|---|---|---|
| Sodium caseinate | 55 | g | High quality protein |
| Calcium caseinate | 17 | g | High quality protein |
| Palm kernel oil | 20 | g | Medium chain |
| | | | triglycerides |
| Fish oil | 16.7 | g | n-3 fatty acids to reduce |
| | | | inflammation |
| Sunflower oil | 5.0 | g | Fatty acids and energy |
| Maltodextrin | 200 | g | Carbohydrate and energy |
| | | | source |
| Yeast extract | 2.26 | g | RNA/nucleotide source |
| Arginine | 18.75 | g | Support T-cell function |
| Vitamin premix | 2.5 | g | |
| Mineral premix | 1.0 | g | |
| Emulsifier | 1.5 | g | |
| Water | 1243 | g | |
| Optional ingredients | 52 | g | As listed elsewhere |

### Example 3

TABLE 3
High protein oral nutritional supplement product, 8 fluid ounces

| Ingredient | Amount | Function |
|---|---|---|
| Milk protein Concentrate | 15 g | High quality protein |
| Oil blend | 6 g | Fatty acids and energy |
| Sucrose | 13 g | Carbohydrate and energy |
| | | source |
| Corn syrup solids | 18.9 g | Carbohydrate and |
| | | sweetness |
| Minerals | 2.5 g | |
| Yeast extract | 1.0 g | RNA/nucleotide source |
| Vitamins | 1.0 g | |
| Water | 200 g | |
| Optional ingredients | | As listed elsewhere |

### Example 4

TABLE 4
Protein and nucleotide modular oral nutrition product for wound healing

| Ingredient | Amount | Function |
|---|---|---|
| Whey protein isolate | 6 g | High quality protein |
| Yeast extract | 2 g | RNA/nucleotide source |
| Citric acid | 1 g | Acidulent |
| Vitamin premix | 0.1 g | |
| Mineral premix | 0.2 g | |
| Optional ingredients | 0.4 g | As listed elsewhere |

### Example 5

TABLE 5
Amino acid and nucleotide modular oral nutrition product for wound
healing

| Ingredient | Amount | Function |
|---|---|---|
| L-arginine | 4 g | High quality protein |
| Yeast extract | 2 g | RNA/nucleotide source |
| Citric acid | 2 g | Acidulent |
| Malic acid | 1 g | Acidulent |
| Vitamin premix | 0.1 g | |
| Mineral premix | 0.2 g | |
| Optional ingredients | 0.4 g | As listed elsewhere |

Nutritional products is preferably understood to further include any number of optional additional ingredients, including conventional food additives, for example one or more, acidulants, additional thickeners, buffers or agents for pH adjustment, chelating agents, colorants, emulsifies, excipient, flavor agent, mineral, osmotic agents, a pharmaceutically acceptable carrier, preservatives, stabilizers, sugar, sweeteners, texturizers, and/or vitamin. The optional ingredients can be added in any suitable amount.

In a preferred embodiment the nutritional composition comprising a high protein component and an exogenous nucleotide. In a further embodiment, the high protein component includes at least one of proteins, peptides, and amino acids, and precursors and metabolites of proteins, peptides, and amino acids in an amount that provides at least about 18% of the total calories of the nutritional composition. In a further embodiment, the nutritional composition has a total volume of 300 mL or less and wherein the high protein component includes at least one of proteins, peptides, and amino acids, and precursors and metabolites of proteins, peptides, and amino acids in an amount greater than about 14 grams. In a further embodiment the high protein component includes at least about 5 grams of protein, at least about 3 grams of a bitter tasting amino acid, and at least about 7 grams of a neutral tasting acid. In a further embodiment, the bitter tasting amino acid is selected from the group consisting of arginine, phenylalanine, tyrosine, leucine, isoleucine, valine, methionine, histidine and combinations thereof. In a further embodiment the neutral tasting acid is selected from the group consisting of glutamine, glycine, alanine, threonine, praline, serine and combinations thereof. In a further embodiment, the exogenous nucleotide is in all amount of about 1 gram/1000 calories of the nutritional composition. In a further embodiment, the exogenous nucleotide is in a monomeric form selected from the group consisting of 5′ Adenosine Monophosphate, 5′-Guanosine Monophosphate, 5′-Cytosine Monophosphate, 5′-Uracil Monophosphate, 5′-Inosine Monophosphate, 5′-Thymine Monophosphate and combinations thereof. In a further embodiment, the exogenous nucleotide is intact ribonucleic acid and/or other forms that contain nucleotides.

In a preferred embodiment, the nutritional composition further comprises an ingredient selected from the group consisting of non-replicating bacteria, fatty acids, triglycerides, lactowolfberry, antioxidants, vitamins, minerals, polyphenolics, flavonoids, EGCg, pycnogenol, α- and β-glucans, alpha-hydroxyisocaproate, aloe, honey, amino acids, branched chain amino acids and combinations thereof.

In a preferred embodiment, the nutritional composition is in an administerable form selected from the group consisting of pharmaceutical formulations, nutritional formulations, dietary supplements, functional foods and beverage products.

In a preferred embodiment, the nutritional composition further comprises antioxidants. In a further embodiment, the antioxidants are selected from a group consisting of: carotenoids, coenzyme Q10 (“CoQ10”), flavonoids, glutathione Goji (Wolfberry) hesperidine, Lactowolfberry, lignan, lutein, lycopene, polyphenols, selenium, vitamin A, vitamin B1, vitamin B6, vitamin B12, vitamin C, vitamin D, vitamin E, or combinations thereof.

In a preferred embodiment, the nutritional composition further comprises carbohydrate.

In a preferred embodiment, the nutritional composition further comprises fats.

In a preferred embodiment, the nutritional composition further comprises fish oils. In a further embodiment, the fish oils are selected from a group consisting of: docosahexaenoic acid (“DHA”) and eicosopentaenoic add (“EPA”).

In a preferred embodiment, the nutritional composition further comprises minerals. In a further embodiment, the minerals are selected from a group consisting of boron, calcium, chromium, copper, iodine, iron, magnesium, manganese, molybdenum, nickel, phosphorus, potassium, selenium, silicon, tin, vanadium, zinc, and combinations thereof.

In a preferred embodiment, the nutritional composition further comprises phytonutrients. In a further embodiment, the phytonutrients are selected from a group consisting of flavonoids and allied phenolic and polyphenolic compounds, terpenoids including carotenoids, and alkaloids; including curcumin, limonin, and quercetin and combinations thereof.

In a preferred embodiment, the nutritional composition further comprises prebiotics. In as further embodiment, the prebiotics are selected from a group consisting of acacia gum, alpha glucan, arabinogalactans, beta glucan, dextrans fructooligosaccharides, galactooligosaccharides, galactomannans, gentiooligosaccharides, glucooligosaccharides, guar gum, inulin, isomaltooligosaccharides, lactosucrose, lactulose, levan, maltodextrins, partially hydrolyzed guar gum, pecticoligosaccharides, retrograded starch, soyoligosaccharides, sugar alcohols, xylooligosaccharides, or a combination thereof.

In a preferred embodiment, the nutritional composition further comprises probiotics. In a further embodiment, the probiotics are selected from a group consisting of, replications and non-replicating: Aerococcus, Aspergillus, Bacteroides, Bifidobacterium, Candida, Clostridium, Debaromyces, Enterococcus, Fusobacterium, Lactococcus, Leuconostoc, Melissococcus, Micrococcus, Mucor, Oenococcus, Pediococcus, Penicillium, Peptostrepococcus, Pichia, Propionibacterium, Pseudocatenlatum, Rhizopus, Saccharomyces, Staphylococcus, Streptococcus, Torulopsis, Weissella, or a combination thereof.

In a preferred embodiment, the nutritional composition further comprises vitamins. In a further embodiment, the vitamins are selected from a group consisting of fat-soluble or water-soluble organic substances including vitamin A, vitamin B1, vitamin B2, vitamin B6, vitamin B12, vitamin C, vitamin D, vitamin E, vitamin K (including Vitamin K1 and Vitamin K1), folic acid and biotin, pro-vitamins, derivatives, analogs. And combinations thereof

In a preferred embodiment, the nutritional composition is complete nutrition.

In a preferred embodiment, the nutritional composition further comprises is incomplete nutrition.

In a preferred embodiment, the method of making a nutritional composition, the method comprises adding a high protein component and an exogenous nucleotide to as nutritional composition. In a further embodiment, the high protein component includes at least one of proteins, peptides, and amino acids, and precursors and metabolites of proteins, peptides, and amino acids in an amount that provides at least 18% of the total calories of the nutritional composition. In a further embodiment, the nutritional composition has a total volume of 300 mL or less and wherein the high protein component includes at least one of proteins, peptides, and amino acids, and precursors and metabolites of proteins, peptides, and amino acids in an amount greater than about 14 grams. In a further embodiment, the high protein component includes at least about 5 grams of protein, at least about 3 grams of a bitter tasting amino acid, and at least about 7 grams of as neutral tasting acid. In a further embodiment, the nutritional composition is in an administrable form selected from the group consisting of pharmaceutical formulations, nutritional formulations, dietary supplements, functional foods and beverage products.

In a further embodiment, the method of improving wound healing or treating a wound in a mammal includes administering to a mammal having a wound a nutritional composition comprising an effective amount of a high protein component and an exogenous nucleotide. In a further embodiment, the nutrition composition is administered to provide the exogenous nucleotide in an amount ranging front about 0.2 g/day to about 4 grams/day. In a further embodiment, the mammal is a human. In a further embodiment, the mammal is elderly. In a further embodiment, the mammal is in a hospital. In a further embodiment, the mammal is in a care facility. In a further embodiment, the mammal is in a nursing home. In a further embodiment, the nutritional composition is a tube feed. In a further embodiment, the administering is for long-term administration. In a further embodiment, the administering is for short-term administration. In a further embodiment, the high protein component includes at least one of proteins, peptides, and amino acids, and precursors and metabolites of proteins, peptides, and amino acids in an amount that provides at least 18% of the total calories of the nutritional composition. In a further embodiment, the nutritional composition has a total volume of 300 mL or less and wherein the high protein component includes at least one of proteins, peptides, and amino acids, and precursors and metabolites of proteins, peptides, and amino acids in an amount greater than about 14 grams. In a further embodiment, the high protein component includes at least about 5 grams of protein, at least about 3 grams of a bitter tasting amino acid, and at least about 7 grams of a neutral tasting acid. In a further embodiment, the wound is a pressure ulcers, surgical incisions, cuts, scrapes and combinations thereof. In a further embodiment, the mammal has diabetes. In a further embodiment, the mammal has renal failure. In a further embodiment, the mammal has hepatic insufficiency or hepatic failure.

In a preferred embodiment, is the method of treating pressure ulcers in a mammal, the method including administering to a mammal having a pressure ulcer a nutritional composition comprising an effective amount of a high protein component and an exogenous nucleotide. In a further embodiment, the nutrition composition is administered to provide the exogenous nucleotide in an amount ranging from about 0.2 g/day to about 4 grams/day. In a further embodiment, the high protein component includes at least one of proteins peptides, and amino acids, and precursors and metabolites of proteins, peptides, and amino acids in an amount that provides at least 18% of the total calories of the nutritional composition. In a further embodiment, the nutritional composition has a total volume of 300 mL or less and wherein the high protein component includes at least one of proteins, peptides, and amino acids, and precursors and metabolites of proteins, peptides, and amino acids in an amount greater than about 14 grams. In a further embodiment, the high protein component includes at least about 5 grams of protein, at least about 3 grams of a bitter tasting amino acid, and at least about 7 grams of a neutral tasting acid. In a further embodiment, the mammal has diabetes. In a further embodiment, the mammal has renal failure. In a further embodiment, the mammal has hepatic insufficiency or hepatic failure. In a further embodiment, the mammal is a human. In a further embodiment, the mammal is elderly. In a further embodiment, the mammal is in a hospital. In a further embodiment, the mammal is in a care facility. In a further embodiment, the mammal is in a nursing home. In a further embodiment, the nutritional composition is a tube feed. In a further embodiment, the administering is for long-term administration. In a further embodiment the administering is for short-term administration.

In a preferred embodiment, is the method of treating surgical incisions in a mammal, the method including administering to a mammal having a pressure ulcer a nutritional composition comprising an effective amount of a high protein component and an exogenous nucleotide. In a further embodiment, the nutrition composition is administered to provide the exogenous nucleotide in an amount ranging from about 0.2 g/day to about 4 grams/day. In a further embodiment, the high protein component includes at least one of proteins, peptides, and ammo acids, and precursors and metabolites of proteins, peptides, and amino acids in an amount that provides at least 18% of the total calories of the nutritional composition. In a further embodiment, the nutritional composition has as total volume of 300 mL or less and wherein the high protein component includes at least one of proteins, peptides, and amino acids, and precursors and metabolites of proteins, peptides, and amino acids in an amount greater than about 14 grams. In a further embodiment, the high protein component includes at least about 5 grams of protein, at least about 3 grains of a bitter tasting amino acid, and at least about 7 grams of a neutral tasting acid. In a further embodiment, the mammal has diabetes. In a further embodiment, the mammal has renal failure. In a further embodiment the mammal has hepatic insufficiency or hepatic failure. In a further embodiment, the mammal is a human. In a further embodiment, the mammal is elderly. In a further embodiment, the mammal is in a hospital. In a further embodiment the mammal is in a care facility. In a further embodiment, the mammal is in a nursing home. In a further embodiment, the nutritional composition is a tube feed. In a further embodiment, the administering is for long-term administration. In a further embodiment, the administering is for short-term administration.

In a preferred embodiment, is wherein the method leads to a decrease in healthcare spending costs. In a further embodiment the decrease in healthcare spending costs is due to decreased length of stay in a hospital. In a further embodiment, the decrease in healthcare spending costs is due to decreased length of stay in a care facility. In a further embodiment, the decrease in healthcare spending costs is due to decreased complications.

All dosage ranges contained within this application are intended to include all numbers, whole or fractions, contained within said range.

It should be understood that various changes and modifications to the presently preferred embodiments described herein will be apparent to those skilled in the art. Such changes and modifications can be made without departing from the spirit and scope of the present subject matter and without diminishing its intended advantages. It is therefore intended that such changes and modifications be covered by the appended claims.

1. A nutritional composition comprising a high protein component and an exogenous nucleotide, the high protein component includes at least one component selected from the group consisting of proteins, peptides, and amino acids, and precursors and metabolites of proteins, peptides, and amino acids in an amount that provides at least about 18% of the total calories of the nutritional composition.

2. The nutritional composition of claim 1, wherein the nutritional composition has a total volume of not greater than 300 mL and the high protein component comprises more than 14 grams.

3. The nutritional composition of claim 1, wherein the high protein component includes at least 5 grams of protein, at least 3 grams of a bitter tasting amino acid, and at least 7 grams of a neutral tasting acid,
the bitter tasting amino acid is selected from the group consisting of arginine, phenylalanine, tyrosine, leucine, isoleucine, valine, methionine, histidine and combinations thereof, and the neutral tasting acid is selected from the group consisting of glutamine, glycine, alanine, threonine, proline, serine and combinations thereof.

4. The nutritional composition of claim 1, wherein the exogenous nucleotide comprises about 1 gram/1000 calories of the nutritional composition.

5. The nutritional composition of claim 1, wherein the exogenous nucleotide is in a monomeric form selected from the group consisting of 5′ Adenosine Monophosphate, 5′-Guanosine Monophosphate, 5′-Cytosine Monophosphate, 5′-Uracil Monophosphate, 5′-Inosine Monophosphate, 5′-Thymine Monophosphate and combinations thereof.

6. The nutritional composition of claim 1, wherein the exogenous nucleotide is intact ribonucleic acid and/or other forms that contain nucleotides.

7. The nutritional composition of claim 1 comprising an ingredient selected from the group consisting of non-replicating bacteria, probiotics, fatty acids, triglycerides, Goji, lactowolfberry, antioxidants, vitamins, minerals, polyphenolics, flavonoids, EGCg, pycnogenol, α- and β-glucans, alpha-hydroxyisocaproate, aloe, honey, amino acids, branched chain amino acids, carbohydrates, fats, fish oils, phytonutrients, prebiotics, and combinations thereof.

8. A method of treating a wound or improving wound healing in a mammal, the method comprising administering to a mammal having a wound a nutritional composition comprising an effective amount of a high protein component and an exogenous nucleotide.

9. The method of claim 8, wherein the nutritional composition comprises the high protein component includes at least one component selected from the group consisting of proteins, peptides, and amino acids, and precursors and metabolites of proteins, peptides, and amino acids in an amount that provides at least about 18% of the total calories of the nutritional composition.

10. The method of claim 8, wherein the nutrition composition is administered to provide the exogenous nucleotide in an amount ranging from about 0.2 g/day to about 4 grams/day.

11. The method claim 8, wherein the mammal is elderly.

12. The method of claim 8, wherein the mammal is in a setting selected from the group consisting of a hospital, care facility, nursing home, and undergoing in-home care.

13. The method of claim 8, wherein the nutritional composition is a tube feed composition.

14. The method of claim 8, wherein the mammal has at least one condition selected from the group consisting of diabetes, renal failure, hepatic insufficiency, and hepatic failure.

15. The method of claim 8, wherein the wound is a pressure ulcers.

16. The method of claim 8, wherein the wound is a surgical incision.

17. The method of claim 8, wherein the method leads to a decrease in healthcare spending costs.

18. The method of claim 17, wherein the decrease in healthcare spending costs is due to decreased length of stay in a hospital.

19. The method of claim 17, wherein the decrease in healthcare spending costs is due to decreased length of stay in a care facility.

20. The method of claim 17, wherein the decrease in healthcare spending costs is due to decreased complications.

21. The method of claim 20, wherein the complications are selected from a group consisting of local infection, septicemia, delayed healing, embolism, thrombosis, iatrogenic incident, hypoxia, and combinations thereof.
